(19) United States
(12) Patent Application Publication
(10) Pub. No.: US 2015/0098059 A1
(43) Pub. Date: Apr. 9, 2015

(54) PORTABLE PUPIL DETECTION DEVICE WITH MULTIBAND STIMULATING LIGHT AND INFRARED ILLUMINATION

(71) Applicants: NATIONAL TAIWAN UNIVERSITY HOPSITAL HSIN-CHU BRANCH, Hsinchu City (TW); NATIONAL CHIAO TUNG UNIVERSITY, Hsinchu City (TW)

(72) Inventors: MANG OU-YANG, HSINCHU CITY (TW); JIN-CHERN CHIOU, HSINCHU CITY (TW); MEI-LAN KO, HSINCHU CITY (TW); TING-WEI HUANG, TAIPEI CITY (TW); JYUN-WEI JHUANG, HSINCHU CITY (TW); CHUN-CHENG HU, TAINAN CITY (TW)

(73) Assignees: NATIONAL TAIWAN UNIVERSITY HOPSITAL HSIN-CHU BRANCH, Hsinchu City (TW); NATIONAL CHIAO TUNG UNIVERSITY, Hsinchu City (TW)

(21) Appl. No.: 14/191,890

(22) Filed: Feb. 27, 2014

(30) Foreign Application Priority Data
Oct. 4, 2013 (TW) 102135988

(51) Int. Cl. A61B 3/11; A61B 3/14

(52) U.S. Cl. CPC A61B 3/112; A61B 3/14
USPC 351/210

(57) ABSTRACT

The present invention discloses a portable pupil detection device with multiband stimulating light and infrared illumination, which comprises at least one pupil detection module for detecting the variation of the pupil of the testee. Each pupil detection module includes an illuminating light source emitting invisible light beams; a stimulating light source emitting visible light beams; at least one beamsplitter corresponding to the illuminating light source, the stimulating light source and an eye of the testee, cooperating with the illuminating light source and the eye to form a first optical path, and cooperating with the stimulating light source and the eye to form a second optical path; a lens corresponding to the beamsplitter and receiving the invisible light beams sent out by the beamsplitter to form an image of the eye; and an imaging element receiving the image of the eye from the lens.

This application claims priority for Taiwan patent application no. 102135988 filed at Oct. 4, 2013, the content of which is incorporated by reference in its entirely.

### BACKGROUND OF THE INVENTION

1. Field of the Invention

The present invention relates to a pupil detection device, particularly to a portable pupil detection device with multiband stimulating light and infrared illumination.

2. Description of the Related Art

The pupil detection device is an instrument detecting the size of the pupil. The pupil detection device uses light to stimulate the pupil and records the variation of the pupil contracted or expanded by the stimulating light for early diagnosing ophthalmic nerve disorders and diabetes.

The conventional pupil detection devices need a plurality of LEDs (Light Emitting Diodes) and beamsplitters and thus have disadvantages of numerous components, high cost and complicated optical paths. Further, the conventional pupil detection devices need precisely calibrating to obtain clear images of the eyeballs. Some conventional pupil detection devices lack a stimulating light source, directly detecting the contraction and expansion of the pupil under natural light. However, the pupil would not contract or expand obviously under natural light. Therefore, the performance of the conventional pupil detection devices lacking a stimulating light source is poor. Although some conventional pupil detection devices have a stimulating light source, they lack a multiband stimulating light source but use white light to illuminate the eye. Thus, they cannot obtain sufficient information.

Accordingly, the present invention proposes a portable pupil detection device with multiband stimulating light and infrared illumination to overcome the abovementioned problems.

### SUMMARY OF THE INVENTION

The primary objective of the present invention is to provide a portable pupil detection device with multiband stimulating light and infrared illumination, which uses a stimulating light source to emit visible light beams to stimulate the pupil and make the pupil contact and expand, and which images the variation of the pupil on an imaging element.

Another objective of the present invention is to provide a portable pupil detection device with multiband stimulating light and infrared illumination, which uses an illuminating light source to emit invisible light beams to detect the pupil, wherein the invisible light beams reflected by the pupil and the images formed thereby are received by an imaging element.

A further objective of the present invention is to provide a portable pupil detection device with multiband stimulating light and infrared illumination, which needs only a stimulating light source for stimulating the eyeball, an illuminating light source for detecting the eyeball, a beamsplitter reflecting light or allowing light to pass, a lens and an imaging element, wherefore the present invention has advantages of fewer components and lower cost.

To achieve the abovementioned objectives, the present invention proposes a portable pupil detection device with multiband stimulating light and infrared illumination. The portable pupil detection device of the present invention is a head-mounted device comprising a monocular or binocular pupil detection module for detecting the variation of the pupil or pupils of a testee. Each pupil detection module includes an illuminating light source, a stimulating light source, at least one beamsplitter, a lens and an imaging element. The illuminating light source and the stimulating light source respectively emit invisible light beams and visible light beams. The stimulating light source is an adjustable multiband light source. The beamsplitter is corresponding to the illuminating light source, the stimulating light source and the eye of the testee. The beamsplitter cooperates with the illuminating light source and the eye to form a first optical path and cooperates with the stimulating light source and the eye to form a second optical path. The beamsplitter transmits the invisible light beams reflected by the eye to the lens and forms the image of the eye on the lens. The imaging element receives the image from the lens.

### BRIEF DESCRIPTION OF THE DRAWINGS

FIG. 1A and FIG. 1B are respectively a rear view and a front view of a portable pupil detection device with multiband stimulating light and infrared illumination according to one embodiment of the present invention;

FIG. 2 is a diagram schematically showing a first embodiment of the pupil detection module shown in FIG. 1A;

FIG. 3 is a diagram schematically showing a second embodiment of the pupil detection module shown in FIG. 1A;

FIG. 4 is a diagram schematically showing a third embodiment of the pupil detection module shown in FIG. 1A;

FIG. 5 is a diagram schematically showing a fourth embodiment of the pupil detection module shown in FIG. 1A;

FIG. 6 is a diagram schematically showing a portable pupil detection device with multiband stimulating light and infrared illumination according to another embodiment of the present invention; and

FIG. 7 is a diagram schematically showing a fifth embodiment of the pupil detection module shown in FIG. 6.

### DETAILED DESCRIPTION OF THE INVENTION

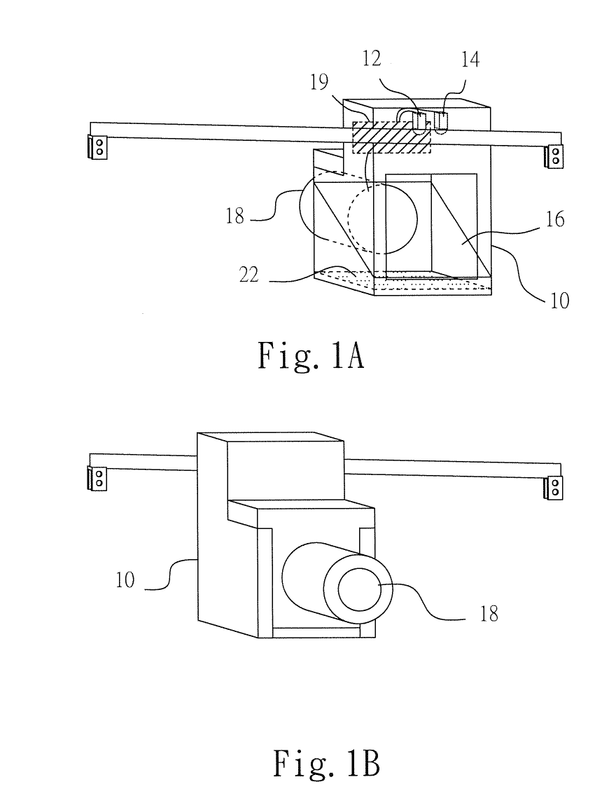

The present invention discloses a portable pupil detection device with multiband stimulating light and infrared illumination. Refer to FIG. 1A and FIG. 1B respectively a rear view and a front view of a portable pupil detection device with multiband stimulating light and infrared illumination according to one embodiment of the present invention. The portable pupil detection device with multiband stimulating light and infrared illumination of the present invention comprises at least one pupil detection module 10. Each pupil detection module 10 includes an illuminating light source 12, a stimulating light source 14, at least one beamsplitter 16, a lens module 18 and a control circuit 19. The lens module 18 has a lens and an imaging element (not shown in the drawing). The illuminating light source 12 is realized by LEDs, a laser device, a fluorescent lamp plus a filter, or an incandescent lamp plus a filter. The illuminating light source 12 emits invisible infrared light beams or invisible ultraviolet light beams. The stimulating light source 14 is realized by LEDs, a laser device, a fluorescent lamp plus a filter, or an incandescent lamp plus a filter. The stimulating light source 14 is an adjustable multiband light source and emits visible light beams having wavelengths of 350-850 nm. The beamsplitter 16 is a beam-splitting mirror, a beam-splitting plate, a prism, or a filter. The beamsplitter 16 is corresponding to the illuminating light source 12, the stimulating light source 14 and the eye of the testee. The beamsplitter 16 cooperates with the eye and the illuminating light source 12 to form a first optical path and cooperates with the eye and the stimulating light source 14 to form a second optical path. The beamsplitter 16 transmits the invisible light beams reflected by the eye to the lens module 18 and forms the image of the eye in the lens module 18. The lens module 18 is a common variable-focus lens module or a variable-focus infrared lens module. The focal length is modified via adjusting the distance between the lens and the imaging element. The imaging element is realized by CMOS (Complementary Metal Oxide Semiconductor) or CCD (Charge Coupled Device). The control circuit 19 is connected with the illuminating light source 12, the stimulating light source 14 and the imaging element of the lens module 18. The control circuit 19 controls the illuminating light source 12 and the stimulating light source 14 to emit light beams, and controls the imaging element to receive images.

Once the pupil detection device has been mounted on the frame of the glasses of the testee, the pupil detection module 10 is exactly aligned to the eye of the testee. In the embodiment shown in FIG. 1A, the pupil detection device merely has a single pupil detection module 10 and thus can only detect the pupil of a single eye. In one embodiment, the pupil detection device has two pupil detection modules 10 and thus can detect the pupils of two eyes simultaneously.

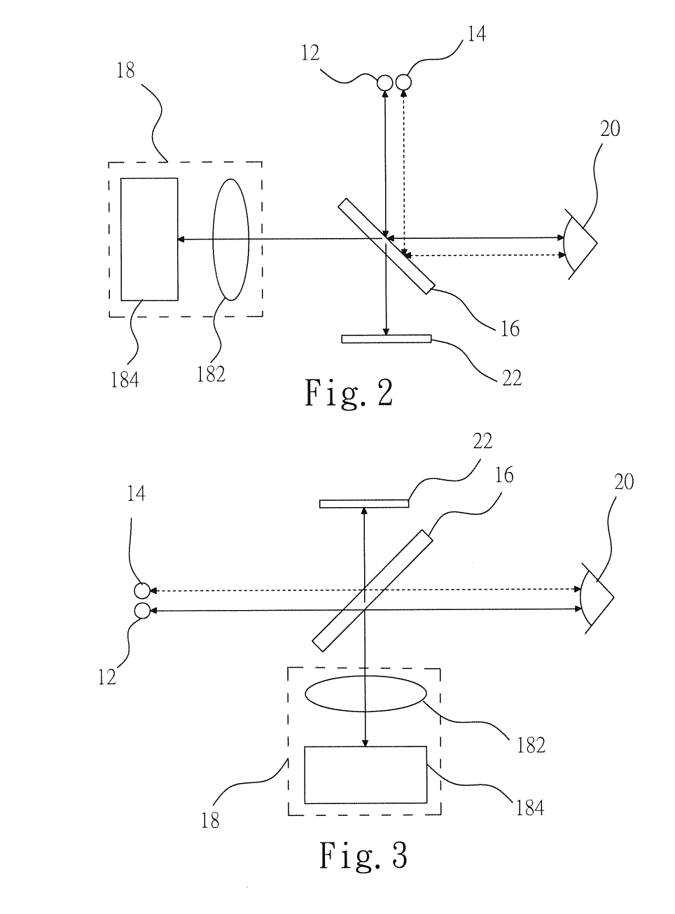

Refer to FIG. 2 a diagram schematically showing a first embodiment of the pupil detection module shown in FIG. 1A. The beamsplitter 16 is arranged between the lens module 18 and an eye 20. The lens module 18 has a lens 182 and an imaging element 184. The illuminating light source 12 and the stimulating light source 14 are arranged above the beamsplitter 16. An absorptive pad 22 is arranged below the beamsplitter 16. In the first embodiment, the beamsplitter 16 reflects the visible light beams emitted by the stimulating light source 14 to the eye 20, and the visible light beams reflected by the eye 20 pass through the beamsplitter 16 and return to the stimulating light source 14; the beamsplitter 16 reflects a portion of the invisible light beams emitted by the illuminating light source 12 to the eye 20, and another portion of the invisible light beams is allowed to pass through the beamsplitter 16 and absorbed by the absorptive pad 22 lest the another portion of the invisible light beams be scattered and affect imaging; the invisible light beams reflected by the eye 20 are partly reflected by the beamsplitter 16 back to the illuminating light source 12 and partly allowed to pass through the beamsplitter 16 and reach the imaging element 184. In the first embodiment, the visible light beams emitted by the stimulating light source 14 are totally reflected to the eye 20 by the beamsplitter 16, and the visible light beams reflected by the eye 20 are totally reflected back to the stimulating light source 14 by the beamsplitter 16.

Refer to FIG. 3 a diagram schematically showing a second embodiment of the pupil detection module shown in FIG. 1A. The beamsplitter 16 is arranged between the eye 20 and the group consisting of the illuminating light source 12 and the stimulating light source 14. The lens module 18 (including the lens 182 and the imaging element 184) is arranged below the beamsplitter 16. The absorptive pad 22 is arranged above the beamsplitter 16. In the second embodiment, the visible light beams emitted by the stimulating light source 14 pass through the beamsplitter 16 and reach the eye 20, and the eye 20 reflects the visible light beams to pass through the beamsplitter 16 and return to the stimulating light source 14; the invisible light beams emitted by the illuminating light source 12 are partly allowed to pass through the beamsplitter 16 and reach the eye 20 and partly reflected to and absorbed by the absorptive pad 22; the invisible light beams reflected by the eye 20 are partly allowed to pass through the beamsplitter 16 and return to the illuminating light source 12 and partly reflected by the beamsplitter 16 to the imaging element 184. In the second embodiment, the beamsplitter 16 allows the visible light beams to pass through totally; the beamsplitter 16 allows the invisible light beams to pass through partly and reflects the invisible light beams partly. In the second embodiment, the absorptive pad 22 also absorbs the scattered light beams for the same reason mentioned above.

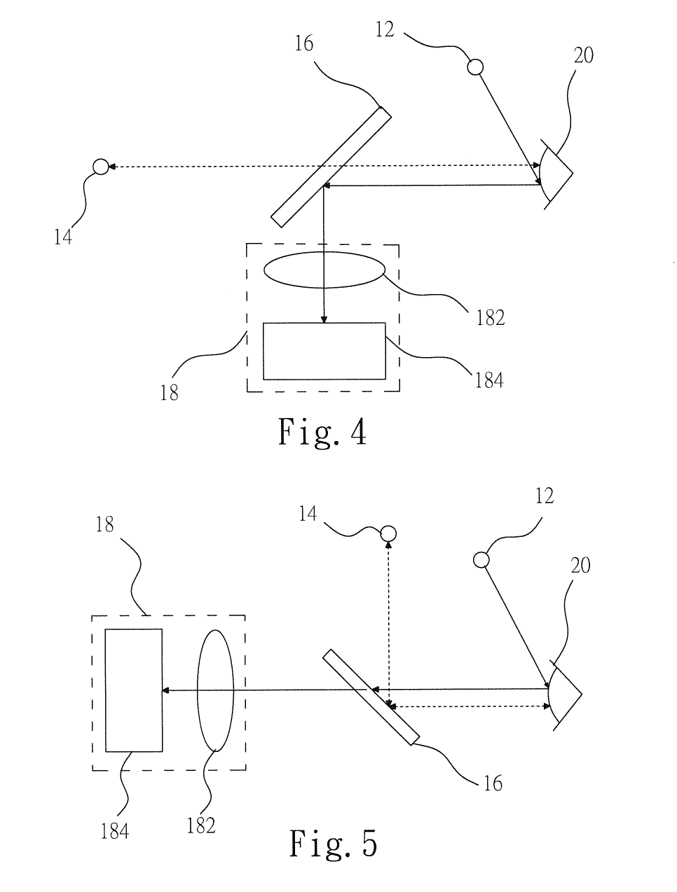

Refer to FIG. 4 a diagram schematically showing a third embodiment of the pupil detection module shown in FIG. 1A. The beamsplitter 16 is arranged among the eye 20, the illuminating light source 12 and the stimulating light source 14. The lens 182 and the imaging element 184 are arranged below the beamsplitter 16. In the third embodiment, the visible light beams emitted by the stimulating light source 14 pass through the beamsplitter 16 and reach the eye 20, and the eye 20 reflects the visible light beams to pass through the beamsplitter 16 and return to the stimulating light source 14; the invisible light beams emitted by the illuminating light source 12 directly reach the eye 20; the invisible light beams reflected by the eye 20 are further reflected by the beamsplitter 16 to the imaging element 184. In the third embodiment, the beamsplitter 16 allows the visible light beams to pass through and reflects the invisible light beams. Therefore, the absorptive pad 22 is unnecessary in the third embodiment.

Refer to FIG. 5 a diagram schematically showing a fourth embodiment of the pupil detection module shown in FIG. 1A. The illuminating light source 12 and the stimulating light source 14 are arranged between the beamsplitter 16 and the eye 20. The lens 182 and the imaging element 184 are arranged beside the beamsplitter 16. In the fourth embodiment, the beamsplitter 16 reflects the visible light beams emitted by the stimulating light source 14 to the eye 20, and the visible light beams reflected by the eye 20 are further reflected by the beamsplitter 16 back to the stimulating light source 14; the invisible light beams emitted by the illuminating light source 12 directly reach the eye 20; the invisible light beams reflected by the eye 20 are allowed to pass through the beamsplitter 16 and reach the imaging element 184. In the fourth embodiment, the beamsplitter 16 reflects the visible light beams and allows the invisible light beams to pass through. Therefore, the absorptive pad 22 is unnecessary in the fourth embodiment.

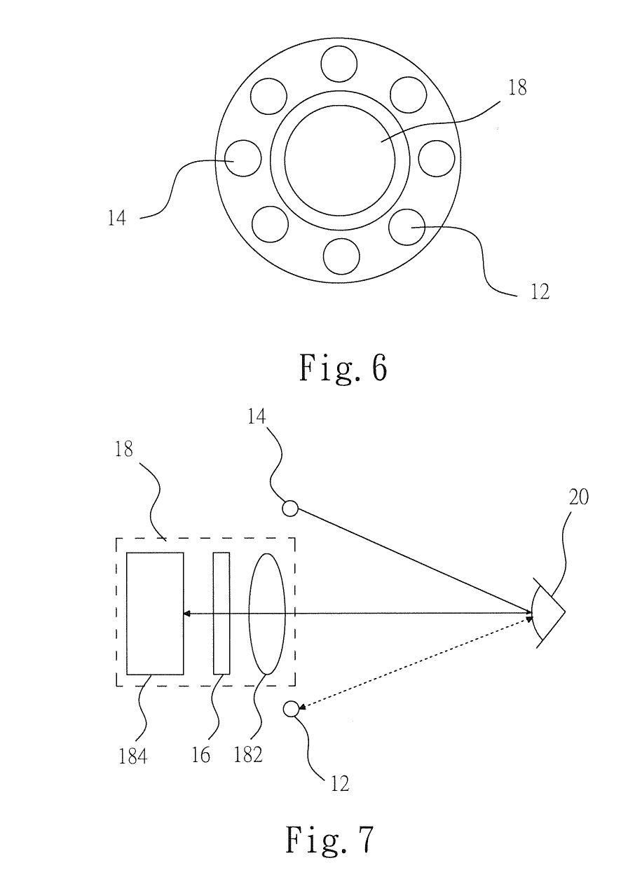

The pupil detection modules of the abovementioned first, second, third and fourth embodiments are all involved in the embodiment shown in FIG. 1A. Refer to FIG. 6 a diagram schematically showing a portable pupil detection device with multiband stimulating light and infrared illumination according to another embodiment of the present invention. Refer to FIG. 7 a diagram schematically showing a fifth embodiment of the pupil detection module shown in FIG. 6. In the fifth embodiment shown in FIG. 7, the imaging element 184, the beamsplitter 16, the lens 182 and the eye 20 are arranged along an identical straight line. The beamsplitter 16 is arranged between the lens 182 and the imaging element 184 or alternatively arranged between the lens 182 and the eye 20. The illuminating light source 12 and the stimulating light source 14 are annularly arranged along the perimeter of the beamsplitter 16, the lens 182 and the imaging element 184. In the present invention, the stimulating light source 14 is a light source emitting multiband light beams. Therefore, a plurality of stimulating light sources is used to respectively emit visible light beams of different frequency bands, in addition to the illuminating light source 12. The visible light beams emitted by the stimulating light sources 14 and the invisible light beams emitted by the illuminating light source 12 are simultaneously projected to the eye 20 and reflected by the eye 20 to the imaging element 184 to form the image of the eye 20.

In the fifth embodiment, the beamsplitter 16 functions as a filter, such as an infrared filter added to a color video camera. Alternatively, the beamsplitter 16 cooperates with the lens 182 and the imaging element 184 to form an infrared lens module.

The portable pupil detection device of the present invention also comprises a control circuit (not shown in the drawings) connected with the stimulating light source, the illuminating light source and the imaging element. The control circuit controls the stimulating light source to emit visible light beams of different frequency bands and different intensities and also controls the illuminating light source to emit invisible light beams of different intensities. The invisible light beams of different intensities and the visible light beams of different frequency bands and different intensities form various combinations of light sources.

Below is introduced an embodiment wherein the beamsplitter filters and splits light beams and wherein the illuminating light source (an infrared LED) emits an invisible infrared light beam having a wavelength (λ) of 900 nm to the beamsplitter. The beamsplitter reflects one half of a light beam and allows another half of the light beam to pass through at the wavelength of 900 nm. Therefore, 50% of the infrared light beam having a wavelength of 900 nm passes through the beamsplitter; 50% of the infrared light beam is reflected by the beamsplitter to the eyeball. The infrared light beam reaching the eyeball is further reflected back to the beamsplitter. The beamsplitter further reflects 50% of the infrared light beam, which was reflected back to the beamsplitter, back to the infrared LED; 50% of the infrared light beam, which was reflected back to the beamsplitter, is allowed to pass through the beamsplitter and received by the imaging element. Thus, the contraction and expansion of the pupil is recorded by the imaging element. The stimulating light source emits visible light beams to the beamsplitter. As the wavelengths of the visible light beams are smaller than 900 nm, almost all of them are reflected to the eyeball, whereby the eyeball is stimulated. Almost all the visible light beams, which were reflected back to the beamsplitter, are reflected back to the stimulating light source. The results of stimulating the eyeball are detected by the light beam emitted by the infrared LED (the illuminating light source).

In conclusion, the present invention proposes a portable pupil detection device with multiband stimulating light and infrared illumination, which comprises a pupil detection module, wherein minimum components are used to construct the pupil detection module, including a lens module containing a lens and an imaging element, a beamsplitter, an adjustable illuminating light source emitting multiband invisible light beams (infrared light beams or ultraviolet light beams) and at least one stimulating light source emitting multiband visible light beams. As fewer components are used in the present invention, the present invention is lightweight and easy to carry about. Therefore, the present invention can be mounted on the glasses of the testee. After the altitude of the pupil detection device and the distance between the eye and the pupil detection device has been adjusted, the pupil detection device can detect the contraction and expansion of the pupil stimulated by the visible light beams. Under illumination of the visible light beams, the eye reflects the invisible light beams to the lens module, whereby the pupil is imaged and the detection results of the pupil are obtained. In one embodiment, the pupil detection device of the present invention comprises two pupil detection modules, whereby the pupils of two eyes of the testee can be detected simultaneously. Moreover, the present invention uses multiband stimulating light beams to acquire more data of the pupil and enhance the diagnosis of related diseases.

The embodiments described above are only to exemplify the present invention but not to limit the scope of the present invention. Any equivalent modification or variation according to the spirit or characteristic of the present invention is to be also included within the scope of the present invention.

What is claimed is:

1. A portable pupil detection device with multiband stimulating light and infrared illumination, which comprises at least one pupil detection module for detecting variation of a pupil of a testee, wherein each said pupil detection module includes
an illuminating light source emitting invisible light beams;
at least a stimulating light source emitting visible light beams;
at least one beamsplitter corresponding to said illuminating light source, said stimulating light source and an eye of said testee, cooperating with said illuminating light source and said eye to form a first optical path, and cooperating with said stimulating light source and said eye to form a second optical path;
a lens corresponding to said beamsplitter and receiving invisible light beams sent out by said beamsplitter to form an image of said eye;
an imaging element receiving said image of said eye from said lens; and
a control circuit controlling operations of said stimulating light source, said illuminating light source and said imaging element.

2. The portable pupil detection device with multiband stimulating light and infrared illumination according to claim 1, wherein said illuminating light source is realized by LEDs (Light Emitting Diodes), a laser device, a fluorescent lamp plus a filter, or an incandescent lamp plus a filter.

3. The portable pupil detection device with multiband stimulating light and infrared illumination according to claim 1, wherein frequencies of invisible light beams emitted by said illuminating light source are in an infrared frequency band or an ultraviolet frequency band.

4. The portable pupil detection device with multiband stimulating light and infrared illumination according to claim 1, wherein each of said illuminating light source and said stimulating light source is coated with a light-homogenizing element.

5. The portable pupil detection device with multiband stimulating light and infrared illumination according to claim 4, wherein said light-homogenizing element is glassine, a diffusion plate, or ground glass.

6. The portable pupil detection device with multiband stimulating light and infrared illumination according to claim 1, wherein said stimulating light source is realized by LEDs (Light Emitting Diodes), a laser device, a fluorescent lamp plus a filter, or an incandescent lamp plus a filter,

7. The portable pupil detection device with multiband stimulating light and infrared illumination according to claim 1, wherein said stimulating light source emits light beams having wavelengths of 350-850 nm.

8. The portable pupil detection device with multiband stimulating light and infrared illumination according to claim 1, wherein said beamsplitter is a beam-splitting mirror, a beam-splitting plate, a prism, or a filter.

9. The portable pupil detection device with multiband stimulating light and infrared illumination according to claim 1, wherein said beamsplitter reflects visible light beams emitted by said stimulating light source to said eye; visible light beams reflected by said eye pass through said beamsplitter and return to said stimulating light source; said beamsplitter reflects a portion of invisible light beams emitted by said illuminating light source to said eye, and another portion of said invisible light beams is allowed to pass through said beamsplitter and absorbed; invisible light beams reflected by said eye is partly reflected by said beamsplitter back to said illuminating light source and partly allowed to pass through said beamsplitter and reach said imaging element.

10. The portable pupil detection device with multiband stimulating light and infrared illumination according to claim 1, wherein visible light beams emitted by said stimulating light source pass through said beamsplitter and reach said eye, and said eye reflects visible light beams to pass through said beamsplitter and return to said stimulating light source; invisible light beams emitted by said illuminating light source are partly allowed to pass through said beamsplitter and reach said eye and partly reflected and absorbed; invisible light beams reflected by said eye are partly allowed to pass through said beamsplitter and return to said illuminating light source and partly reflected by said beamsplitter to said imaging element.

11. The portable pupil detection device with multiband stimulating light and infrared illumination according to claim 1, wherein visible light beams emitted by said stimulating light source pass through said beamsplitter and reach said eye, and said eye reflects visible light beams to pass through said beamsplitter and return to said stimulating light source; invisible light beams emitted by said illuminating light source directly reach said eye; invisible light beams reflected by said eye are further reflected by said beamsplitter to said imaging element.

12. The portable pupil detection device with multiband stimulating light and infrared illumination according to claim 1, wherein said beamsplitter reflects visible light beams emitted by said stimulating light source to said eye, and said visible light beams reflected by said eye are further reflected by said beamsplitter back to said stimulating light source; invisible light beams emitted by said illuminating light source directly reach said eye; invisible light beams reflected by said eye are allowed to pass through said beamsplitter and reach said imaging element.

13. The portable pupil detection device with multiband stimulating light and infrared illumination according to claim 1, wherein said visible light beams and said invisible light beams are simultaneously projected to said eye and then simultaneously reflected to said imaging element by said eye.

14. The portable pupil detection device with multiband stimulating light and infrared illumination according to claim 13, wherein said beamsplitter is arranged before said imaging element or said lens to filter out said visible light beams.

15. The portable pupil detection device with multiband stimulating light and infrared illumination according to claim 1, wherein said imaging element is realized by CMOS (Complementary Metal Oxide Semiconductor) or CCD (Charge Coupled Device).

16. The portable pupil detection device with multiband stimulating light and infrared illumination according to claim 1, wherein said control circuit controls said stimulating light source to emit visible light beams of different frequency bands and different intensities and also controls said illuminating light source to emit invisible light beams of different intensities; said invisible light beams of different intensities and said visible light beams of different frequency bands and different intensities form various combinations of light sources.

17. The portable pupil detection device with multiband stimulating light and infrared illumination according to claim 1 comprising two pupil detection modules for detecting pupils of two eyes.

18. The portable pupil detection device with multiband stimulating light and infrared illumination according to claim 1, wherein said imaging element can vary focal length.

19. The portable pupil detection device with multiband stimulating light and infrared illumination according to claim 8 further comprising an absorptive pad absorbing a portion of said invisible light beams passing through said beamsplitter.

20. The portable pupil detection device with multiband stimulating light and infrared illumination according to claim 9 further comprising an absorptive pad absorbing a portion of said invisible light beams reflected by said beamsplitter.
